(19) United States
(12) Patent Application Publication
(10) Pub. No.: US 2023/0073089 A1
(43) Pub. Date: Mar. 9, 2023

(54) CO-PRODUCTION OF METHANOL, AMMONIA AND UREA

(71) Applicant: Topsoe A/S, Kgs. Lyngby (DK)

(72) Inventors: Emil Andreas Tjärnehov, Limhamn (SE); Pat A. Han, Smørum (DK)

(73) Assignee: Topsoe A/S, Kgs. Lyngby (DK)

(21) Appl. No.: 17/800,151

(22) PCT Filed: Feb. 24, 2021
(86) PCT No.: PCT/EP2021/054517

§ 371 Date: Aug. 16, 2022

(30) Foreign Application Priority Data
Feb. 28, 2020 (DK) PA 2020 00256

(51) Int. Cl. C01C 1/04; C01B 3/38; C07C 29/151

(52) U.S. Cl. CPC C01C 1/0488; C01B 3/382; C07C 29/1518; C01B 2203/0233; C01B 2203/0283; C01B 2203/0445; C01B 2203/068

(57) ABSTRACT

Sequential and once-through (single pass) process for the co-production of methanol and ammonia and conversion of at least a part of ammonia to urea by reaction of the ammonia with carbon dioxide collected from a primary reformer flue gas together with carbon dioxide separated from reformed gas in a carbon dioxide removal stage.

The present invention relates to a process for the co-production of methanol, ammonia and urea from a hydrocarbon feed with reduced emission of carbon dioxide to the atmosphere and flexible control of the amount of methanol, ammonia and urea produced from the feed. More particularly the invention is concerned with a sequential and once-through (single pass) process for the co-production of methanol and ammonia and conversion of at least a part of ammonia to urea by reaction of the ammonia with carbon dioxide collected from a primary reformer flue gas together with carbon dioxide separated from reformed gas in a carbon dioxide removal stage.

Current processes for co-production of methanol and ammonia involve generally parallel processes in which a common reforming section is used to generate a synthesis gas which is split in separate parallel streams, one of which is used for methanol synthesis and the other for ammonia synthesis. The co-production of methanol and ammonia can also be conducted sequentially or in series, where the synthesis gas produced in the reforming section is first converted to methanol and the unreacted gas containing nitrogen and hydrogen is subsequently used for ammonia synthesis.

In a first aspect of the present invention provides a process for co-producing methanol, ammonia and urea in series which process allows a flexible control of the amount of methanol, ammonia and urea product from a given amount of hydrocarbon and which at the same time enables minimum release of carbon dioxide to the atmosphere.

The co-production process produces methanol and ammonia, where ammonia can be used for further production of urea together with CO2. CO2 can be extracted from the co-production process side which will then limit the production of methanol (as methanol is produced from carbon oxides and hydrogen). In order to match the production needs we found that an additional CO2 recovery on the flue gas side can match the CO2 requirement and reduces the CO2 emission. The process can then be controlled to match a methanol demand and a urea (ammonia) demand.

Thus, the present invention is a Process for co-producing methanol, ammonia and urea from a hydrocarbon feedstock, the process comprising the steps of
a) primary and secondary steam reforming of a hydrocarbon feedstock, and obtaining a steam reformed effluent comprising hydrogen, nitrogen, carbon monoxide and carbon dioxide;
b) passing a part of the steam reformed effluent from step (a) to a carbon dioxide removal stage to produce an effluent with a reduced content of carbon dioxide;
c) by-passing the remaining part of the steam reformed effluent the carbon dioxide removal stage and combining the effluent withdrawn from step (b) with the by-passed part of the steam reformed effluent to provide a methanol synthesis gas comprising hydrogen, nitrogen and carbon monoxide and carbon dioxide;
d) adding hydrogen recovered from a downstream ammonia synthesis stage to the methanol synthesis gas obtained in step (c);
e) catalytically converting the methanol synthesis gas in a once-through methanol synthesis step and withdrawing a liquid effluent comprising methanol and a gas effluent comprising nitrogen and hydrogen;
e) catalytically converting the gas effluent withdrawn in step (e) to ammonia in the ammonia synthesis stage; and
f) converting at least a part of the ammonia from step (e) to urea by reaction with carbon dioxide removed in step (b) together with carbon dioxide contained in flue gas recovered from the primary steam reforming in step (a).

As used herein the term “primary reforming” means reforming being conducted in a conventional steam methane reformer (SMR), i.e. tubular reformer with the heat required for the endothermic reforming being provided by radiation heat from burners, such as burners arranged along the walls of the tubular reformer.

As used herein the term “secondary reforming” means reforming being conducted in an autothermal reformer or a catalytic partial oxidation reactor using air or oxygen enriched air.

In the process of the invention, the amount of methanol production is adjusted by the amount of carbon dioxide by-passed the carbon dioxide removal stage. Increasing the amount of carbon dioxide in the methanol synthesis gas with by-passed carbon dioxide results in an increased methanol production and vice versa.

In order to provide the required amount of hydrogen when adding carbon dioxide to the methanol synthesis gas, hydrogen recovered form the ammonia synthesis stage must be added to the synthesis gas, preferably in amount to provide a module M=(H₂—CO₂)/(CO+CO₂) of at least 2.5, such as between 2.5 and 10.

Recovering hydrogen from the ammonia synthesis results in the further advantage of minimizing the primary reformer size and improved utilization of carbon dioxide in the flue gas form the burners of the reformer because of the less heat required in the minimized reformer.

In an embodiment, the amount of hydrogen in the reformed effluent can be further adjusted by means of the water gas shift reaction.

Preferably, the amount of hydrogen added to the methanol synthesis gas in step (d) is adjusted to provide a module M is at least 2.5, such as between 2.5 and 10.

In the present invention, carbon dioxide generated in in the burners is advantageously utilized in the preparation of urea, which decreases the carbon dioxide foot print of the process.

The amount of carbon dioxide recovered from the burner flue gas and from the carbon dioxide removal stage is adjusted to the desired production of urea.

The above measures allow flexible production of methanol, ammonia and urea depending on the actual demand of the producer.

The process of the invention makes direct use of the reactions governing reforming, methanol synthesis and ammonia synthesis so that methanol and ammonia can be co-produced without venting large amounts of carbon dioxide being captured from the synthesis gas. The carbon oxides from the process can be fully utilized for methanol and urea production

Removal of the part of the carbon dioxide contained in the steam reformed effluent is typically obtained by means of highly expensive CO2-removal stages in the form of acid gas wash, such as conventional MDEA and carbonate wash processes.

Thus, a further advantage of the invention is the reduction of the amount of carbon dioxide to be removed, when by-passing a part of the steam reformed effluent the removal stage.

The process may comprise further parallel methanol processes. I.e. one or more additional methanol processes may be worked in the parallel in the methanol synthesis step of the process of the invention. The parallel one, two, three or more parallel methanol processes may be interconnected by one or more synthesis gas line.

Thus, in an embodiment of the invention the once-through methanol synthesis step is performed in parallel methanol production lines.

As used herein, the term “once-through methanol synthesis stage” means that methanol is produced in at least one catalytic reactor operating in a single pass configuration, i.e. without significant recirculation (not more than 5%, i.e. less than 5%, often 0%) of the volume flow of any gas produced in the methanol synthesis back to the at least one methanol reactor of the methanol synthesis stage, particularly the gas effluent containing hydrogen and unconverted carbon oxides.

The process of the present invention is environmentally friendly because there are no emissions to the surroundings of the CO₂ captured from the methanol and ammonia synthesis gas. Practically all carbon monoxide (and carbon dioxide) produced in the process is used for methanol synthesis and the urea synthesis.

The methanol synthesis stage is preferably conducted by conventional means by passing the synthesis gas at high pressure and temperatures, such as 60-150 bars, preferably 120 bars and 150-300° C. through at least one methanol reactor containing at least one fixed bed of methanol catalyst. A particularly preferred methanol reactor is a fixed bed reactor cooled by a suitable cooling agent such as boiling water, e.g. boiling water reactor (BWR).

In a specific embodiment the methanol synthesis stage in step (e) is conducted by passing the synthesis gas through one boiling water reactor and subsequently through an adiabatic fixed bed reactor, or by passing the synthesis gas through a series of boiling water reactors and subsequently through an adiabatic fixed bed reactor.

Since the methanol synthesis stage is once-through, there is no need for recirculation of a part of the overhead fraction from the separator of the adiabatic fixed bed reactor back to the first methanol reactor of the methanol synthesis stage.

When the amount of carbon monoxide in the gas effluent from the methanol synthesis step in step (e) exceeds the amount, which is acceptable for use in the ammonia synthesis stage, the effluent is passed through a methanation step in order to remove carbon monoxide by reaction to methane.

Thus, in an embodiment of the invention, the process comprises the further step of subjecting the gas effluent from step (d) to a methanation reaction upstream step (e).

In step (e) the ammonia synthesis gas optionally from the methanation step containing the right proportion of hydrogen and nitrogen (preferably H₂:N₂ molar ratio of 3:1) is optionally passed through a compressor to obtain the required ammonia synthesis pressure, such as 120 to 200 bar, preferably about 130 bar. Ammonia is then produced in a conventional manner by means of an ammonia synthesis loop. The effluent containing ammonia contains also hydrogen, nitrogen and inerts such as methane and argon. Ammonia may be recovered from the effluent containing ammonia as liquid ammonia by condensation and subsequent separation. Preferably, an off-gas stream containing hydrogen, nitrogen and methane is withdrawn from the ammonia synthesis stage, as also is a hydrogen-rich stream (>90 vol % H₂). These streams may for instance stem from a purge gas recovery unit. This hydrogen stream is added to the methanol synthesis stage, for instance by combining with the methanol synthesis gas. The recycle of this hydrogen-rich stream enables a higher efficiency in the process as useful hydrogen is utilised in the methanol synthesis and subsequent ammonia synthesis rather than simply being used as fuel.

1. Process for co-producing methanol, ammonia and urea from a hydrocarbon feedstock, the process comprising the steps of
a) primary and secondary steam reforming of a hydrocarbon feedstock, and obtaining a steam reformed effluent comprising hydrogen, nitrogen, carbon monoxide and carbon dioxide;
b) passing a part of the steam reformed effluent from step (a) to a carbon dioxide removal stage to produce an effluent with a reduced content of carbon dioxide;
c) by-passing the carbon dioxide removal stage with the remaining part of the steam reformed effluent and combining the effluent withdrawn from step (b) with the by-passed part of the steam reformed effluent to provide a methanol synthesis gas comprising hydrogen, nitrogen and carbon monoxide and carbon dioxide;
d) adding hydrogen recovered from a downstream ammonia synthesis stage to the methanol synthesis gas obtained in step (c);
e) catalytically converting the methanol synthesis gas in a once-through methanol synthesis step and withdrawing a liquid effluent comprising methanol and a gas effluent comprising nitrogen and hydrogen;
f) catalytically converting the gas effluent withdrawn in step (e) to ammonia in the ammonia synthesis stage; and
g) converting at least a part of the ammonia rom step (e) to urea by reaction with carbon dioxide removed in step (b) together with carbon dioxide contained in flue gas recovered from the primary steam reforming in step (a).

2. The process of claim 1, comprising the further step of subjecting the steam reformed effluent from step (a) to a water gas shift reaction.

3. The process of claim 1, comprising the further step of subjecting the gas effluent from step (d) to a methanation reaction upstream step (e).

4. The process of claim 1, wherein the amount of hydrogen added to the methanol synthesis gas in step (d) is adjusted to provide a module M between 2.5 and 10.

5. The process of claim 1, wherein the once-through methanol synthesis step is performed in parallel methanol production lines.
